(19) United States
(12) Patent Application Publication
(10) Pub. No.: US 2012/0226161 A1
(43) Pub. Date: Sep. 6, 2012

(54) METHODS AND SYSTEMS FOR CONFIGURING ULTRASOUND SYSTEMS FOR ULTRASOUND EXAMINATIONS

(75) Inventors: Laurent Pelissier, North Vancouver (CA); Robert B. Thompson, Vancouver (CA)

(73) Assignee: ULTRASONIX MEDICAL CORPORATION, Richmond (CA)

(21) Appl. No.: 13/473,554

(22) Filed: May 16, 2012

Related U.S. Application Data
(63) Continuation of application No. 11/616,814, filed on Dec. 27, 2006.
(60) Provisional application No. 60/744,192, filed on Apr. 3, 2006.

(51) Int. Cl. A61B 8/13

(52) U.S. Cl. 600/443

(57) ABSTRACT

A method for configuring an ultrasound system for an examination comprises displaying a plurality of examination categories for selection by a user, receiving a user-selected examination category, identifying any probes connected to the ultrasound system, determining which of the connected probes are suitable for the user-selected examination category, for each suitable connected probe, displaying an indication identifying the suitable connected probe for selection by the user, receiving a user-selected probe identification, and, activating a selected probe indicated by the user-selected probe identification for data acquisition.

### REFERENCE TO RELATED APPLICATION

This application claims the benefit under 35 U.S.C. §119 of U.S. application Ser. No. 60/744,192 filed 3 Apr. 2006 which is hereby incorporated herein by reference. This application is a continuation of U.S. patent application Ser. No. 11/616,814 filed on 27 Dec. 2006 and entitled METHODS AND SYSTEMS FOR CONFIGURING ULTRASOUND SYSTEMS FOR ULTRASOUND EXAMINATIONS.

### TECHNICAL FIELD

The invention relates to ultrasound examinations. Certain embodiments of the invention provide methods and systems which assist a user in setting up an ultrasound system for an examination.

### BACKGROUND

Ultrasound systems are used to explore internal areas of a subject's body. Ultrasound examinations are non-destructive and versatile and can provide high quality images. Images produced from ultrasound examinations may be useful in diagnosing a wide variety of conditions.

A typical ultrasound system has a transducer, an electronic controller, and a user interface. The transducer typically has several piezoelectric transducer elements coupled to a frame. The transducer may have elements arranged in any of several different geometries, depending upon the medical application for which the transducer will be used. The electronic controller drives the transducer and collects and processes data from the transducer to generate, store, display and manipulate images. The user interface may include various input/output devices which allow a user to control the operation of the ultrasound system.

In today's practices, a user of ultrasound systems may be required to work on a variety of different systems, all with different layouts. In addition to this, use of ultrasound imaging is expanding into new applications in fields where the users may not be experienced with ultrasound systems.

There is a need for methods and systems which facilitate the setup of an ultrasound system for an examination.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a method for configuring an ultrasound system for an examination. The method comprises displaying a plurality of examination categories for selection by a user, receiving a user-selected examination category, identifying any probes connected to the ultrasound system, determining which of the connected probes are suitable for the user-selected examination category, for each suitable connected probe, displaying an indication identifying the suitable connected probe for selection by the user, receiving a user-selected probe identification, and, activating a selected probe indicated by the user-selected probe identification for data acquisition.

Another aspect of the invention provides an ultrasound system comprising a processor, a user interface coupled to the processor, a display coupled to the processor and a memory coupled to the processor. The processor has at least one probe connection associated therewith to which a probe may be connected. The memory has computer-readable instructions stored thereon which, when executed by the processor, cause the processor to execute an examination configuration method. The examination configuration method comprises displaying a plurality of examination categories for selection by a user, receiving a user-selected examination category, identifying any probes connected to the ultrasound system, determining which of the connected probes are suitable for the user-selected examination category, for each suitable connected probe, displaying an indication identifying the suitable connected probe for selection by the user, receiving a user-selected probe identification, and, activating a selected probe indicated by the user-selected probe identification for data acquisition.

In addition to the exemplary aspects and embodiments described above, further aspects and embodiments will become apparent by reference to the drawings and by study of the following detailed descriptions.

### BRIEF DESCRIPTION OF DRAWINGS

Exemplary embodiments are illustrated in referenced figures of the drawings. It is intended that the embodiments and figures disclosed herein are to be considered illustrative rather than restrictive.

In drawings which illustrate non-limiting embodiments of the invention:

FIG. 1 shows an ultrasound system according to one embodiment of the invention.

FIG. 2 schematically depicts elements of the ultrasound system of FIG. 1.

FIG. 3 is a flowchart illustrating a method according to one embodiment of the invention.

FIGS. 4A-E are screen shots from a display of an ultrasound system according to one embodiment of the invention.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The invention provides methods and systems for setting up an ultrasound system for an examination. Certain embodiments guide a user through the process of setting up the ultrasound system. In a system according to one embodiment of the invention, the system presents the user with a choice as to the type of examination to be performed. The system then determines which, if any, of the probes that are connected to the ultrasound system are suitable for performing the type of examination specified by the user. The system then presents the user with a choice as to which of the suitable connected probes are to be used, and then once the user selects a probe, activates the selected probe for data acquisition.

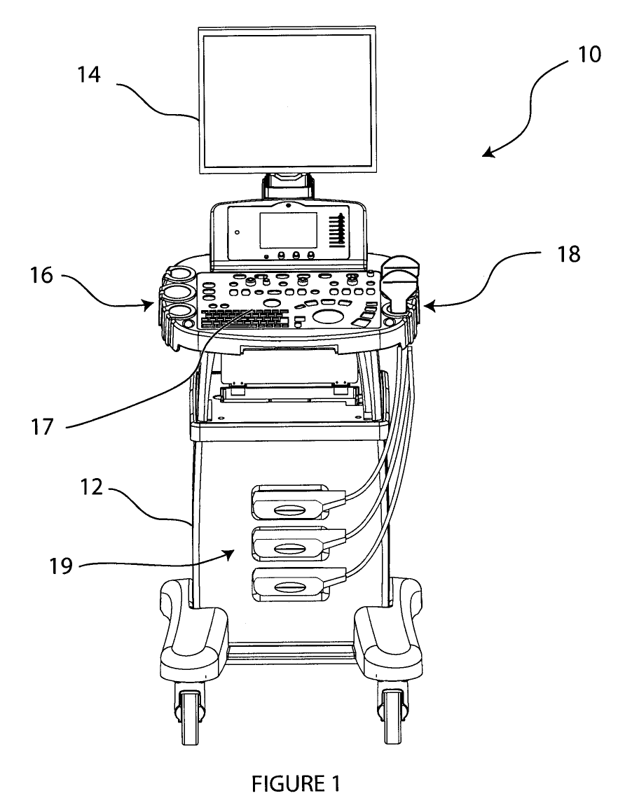

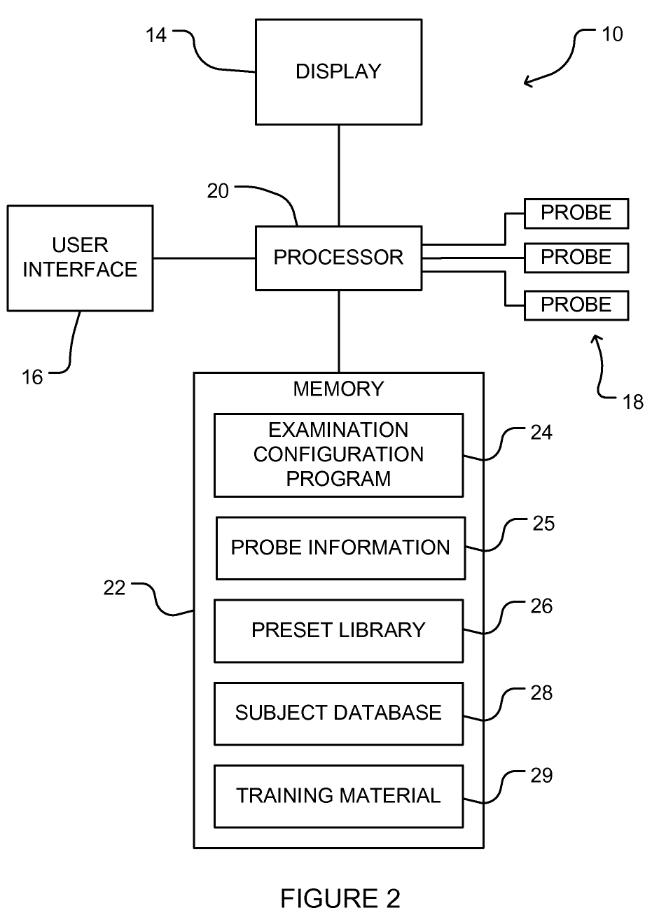

FIG. 1 shows an ultrasound system 10 according to one embodiment of the invention. FIG. 2 schematically depicts various elements of ultrasound system 10. Ultrasound system 10 comprises a housing 12 which houses a processor 20 and a memory 22 (see FIG. 2). Processor 20 is coupled to a display 14 and a user interface 16, which may be mounted atop housing 12. A user of ultrasound system 10 may interact with processor 20 by means of user interface 16. User interface 16 may comprise a quick start button 17 which a user may press to start a method for configuring an ultrasound system for an examination according to one embodiment of the invention.

A plurality of probes 18 are detachably coupled to processor 20 by means of a plurality of probe connections 19 associated with processor 20. Processor 20 is configured to send signals to probes 18 which in turn cause transducers in probes 18 to emit ultrasonic pulses. The ultrasonic pulses from the transducers in probes 18 are directed towards an area of interest in a subject's body. Reflected and/or scattered ultrasonic vibrations (which are sometimes referred to as “echoes”) are received by the transducers in probes 18. The transducers in probes 18 convert the received echoes into electrical signals which are provided to processor 20. Processor 20 receives the electrical signals and converts the electrical signals into image data. The image data may be provided to display 14 in real time, and/or may be stored in memory 22 or provided to an external system (not shown). The operation of processor 20 may be controlled by software stored in memory 22.

Memory 22 has an examination configuration program 24 stored therein. Examination configuration program 24 comprises instructions which, when executed by processor 20, implement a method for configuring ultrasound system 10 for an ultrasound examination according to one embodiment of the invention. Memory 22 may also have probe information 25 stored therein. Probe information 25 may comprise, for example, a plurality of probe identifications, and a suitability indication for each of a plurality of examination categories for each of the plurality of probe identifications.

A preset library 26 and a subject database 28 may also be stored in memory 22 to facilitate configuration of an ultrasound system for an examination, as described further below. Preset library 26 may comprise a plurality of sets of operational parameters for probes 18. Subject database 28 may comprise information identifying a plurality of examination subjects. Alternatively, processor 20 may be in communication with an external database (not shown) wherein sets of operational parameters and subject data are stored. Memory 22 may also have additional software (not shown) stored therein for controlling conventional setup and operation ultrasound system 10.

Memory 22 may also have training material 29 stored therein. Training material 29 may comprise, for example, information relating to the use of each probe identified by the plurality of probe identifications for each of the plurality of examination categories. Training material 29 may comprise videos, pictures, text, or other material.

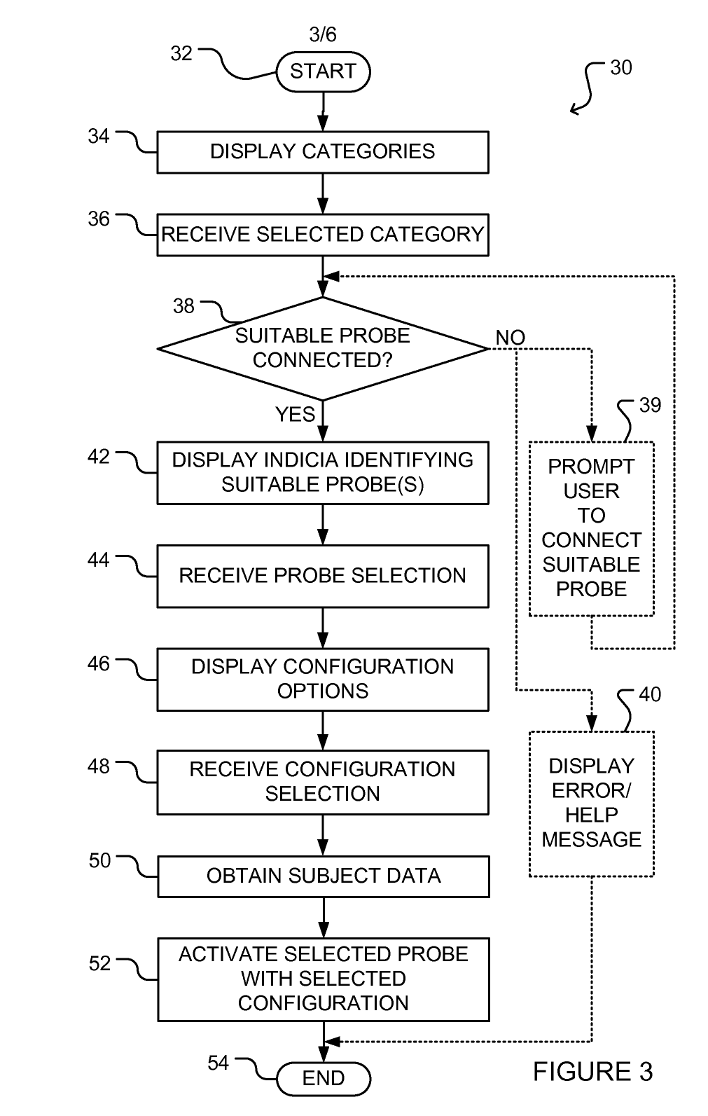

FIG. 3 illustrates a method 30 of configuring an ultrasound system such as ultrasound system 10 for an ultrasound examination according to one embodiment of the invention. Method 30 begins at block 32 in response to input from a user of an ultrasound system. The input which causes method 30 to begin may comprise, for example, pressing a dedicated button on a keyboard which forms part of the user interface of the system to be setup for the examination. The user could also start method 30 by performing any other suitable action, such as selecting an icon on a display with a pointing device, pressing an area of a touch sensitive display, speaking a start command word recognizable to voice recognition software, etc.

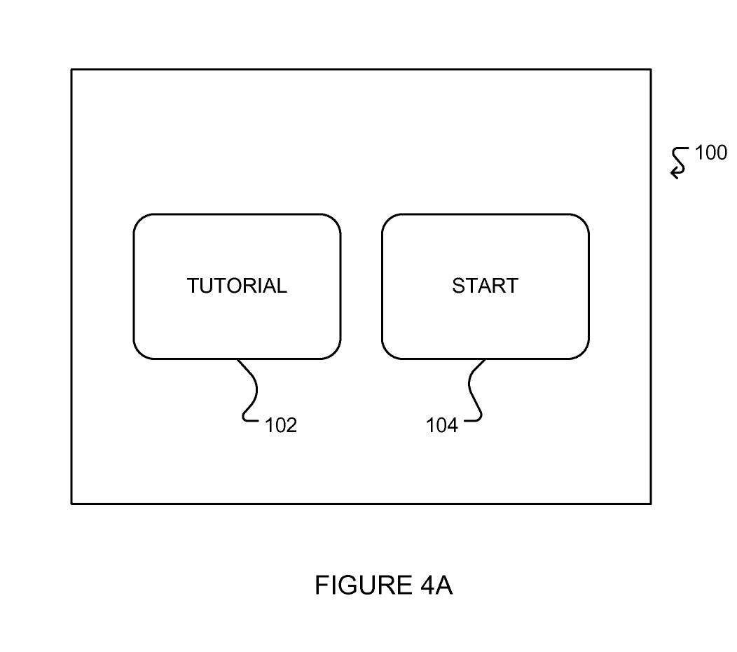

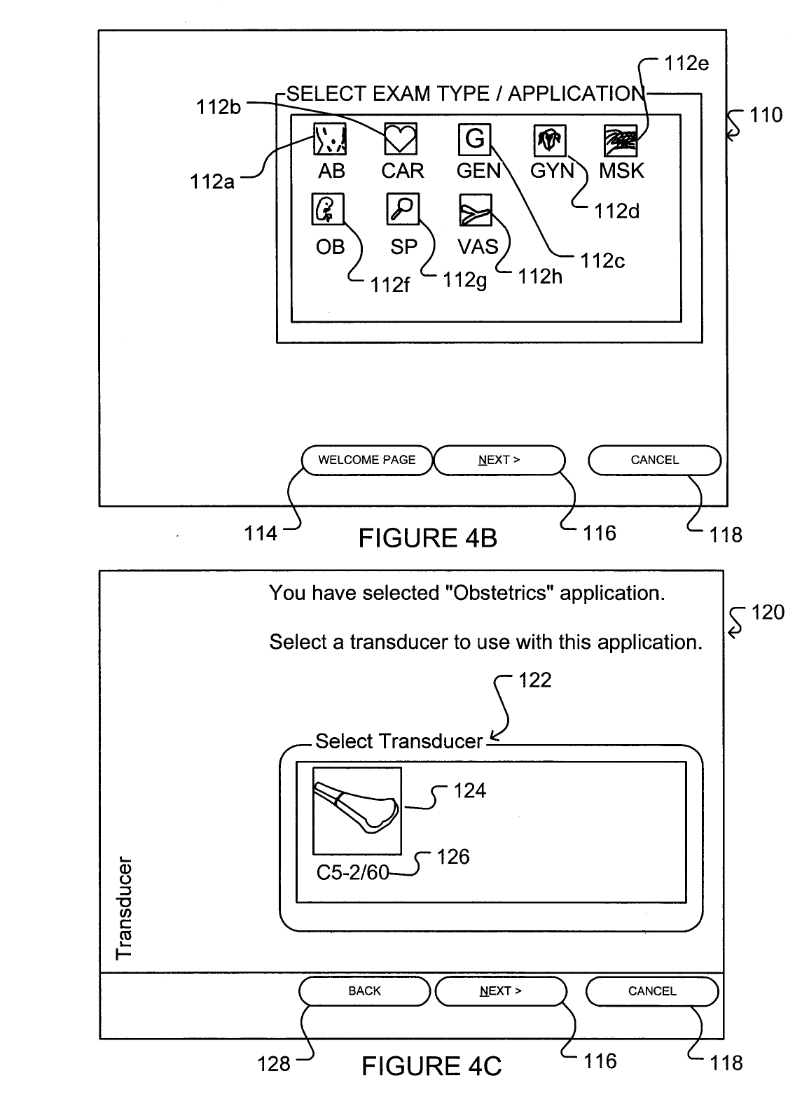

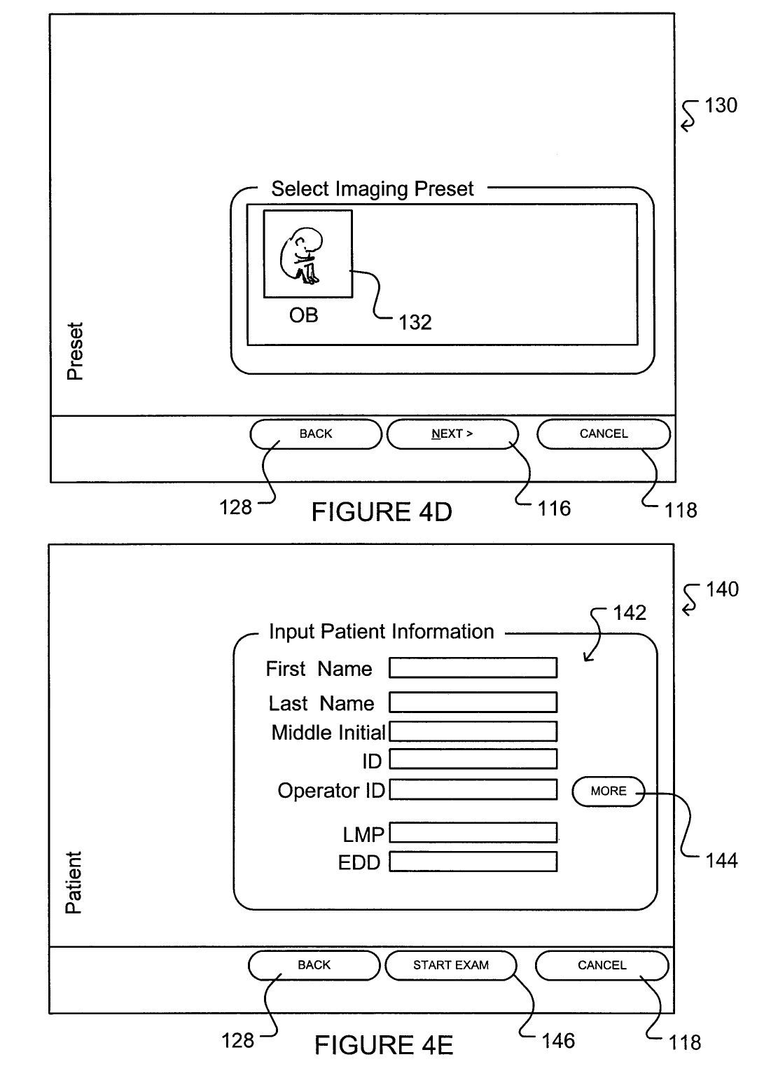

A welcome page may be displayed to the user at block 32. FIG. 4A shows an example screen shot 100 from a screen of a display such as display 14 wherein a welcome page is displayed, the welcome page including a TUTORIAL button 102 and a START button 104. The user may view informational material about the ultrasound system by pressing TUTORIAL button 102. For example, when the user presses TUTORIAL button 102, the ultrasound system may enter into a tutorial mode wherein the user may cause informational material about each control of the user interface to be displayed by touching that control. In tutorial mode, the system may also provide the user with the option to view a video, flash presentation, or other material which provides general application about the ultrasound system and how it may be used. Alternatively, in some embodiments a video, flash presentation, or other material which provides general application about the ultrasound system and how it may be used may automatically be displayed to the user. The user may proceed to configure the ultrasound system by pressing START button 104, and proceeding to block 34. Alternatively, method may proceed directly to block 34 without displaying a welcome page.

At block 34, one or more categories are displayed to the user. FIG. 4B shows an example screen shot 110 from a screen of a display such as display 14 wherein eight categories 112a-h are displayed. In the illustrated example, categories 112a-h respectively comprise Abdominal (AB), Cardiological (CAR), General (GEN), Gynecological (GYN), Musculo-skeletal (MSK), Obstetrics (OB), Small Parts (SP) and Vascular (VAS) examination categories.

At block 34, the user may also be presented with the option to refer to training material such as tutorials and the like. For example, the user could press a WELCOME PAGE button 114 to cause the screen to display the welcome page.

Method 30 proceeds to block 36 when a selected category is received from the user. In the illustrated example, the user selects a category and proceeds to block 36 by highlighting category 112f and pressing a NEXT button 116. The user may also terminate method 30 at any time by pressing a CANCEL button 118.

At block 38 the processor determines whether any suitable probes for the selected category are connected to the ultrasound system. The processor may obtain information about the types of any probes connected to the ultrasound system, for example, by querying the probes themselves or by consulting a data structure stored in memory which is updated to reflect disconnections and reconnections of probes to the ultrasound system. The processor may obtain information about the types of probes which would be suitable for the selected category by consulting a data structure stored in memory which indicates probes suitable for each of a plurality of examination categories.

If no suitable probes are connected to the ultrasound system (block 38 NO output), method may optionally proceed to either block 39 or block 40. At block 39 the user is prompted to connect a suitable probe and method 30 returns to block 38. At block 40 a suitable help and/or error message is displayed, and method 30 proceeds to block 54 and ends.

If at least one suitable probe is connected to the ultrasound system (block 38 YES output), method proceeds to block 42. At block 42 indicia identifying the suitable connected probe(s) are displayed to the user. FIG. 4C shows an example screen shot 120 wherein indicia 122 are displayed, identifying a single suitable connected probe. If there were one or more other suitable connected probes, additional indicia would be displayed for each such probe. Indicia 122 may comprise, for example, a probe icon 124 and a probe name 126. Probe icon 124 may include, for example, a picture of the associated probe. It is to be understood that indicia 122 could alternatively comprise other features.

Method 30 proceeds to block 44 when input identifying a selected probe is received from the user. The user may select a probe and proceed to block 44, for example, by highlighting an indicia which identifies a desired probe and pressing NEXT button 116. The user may also cause method to return to block 34 to select a different examination category by pressing BACK button 128.

At block 46 one or more configurations are displayed to the user for selection. Each configuration may comprise a set of operational parameters for the selected probe and the selected category. The sets of operational parameters may be stored in a data structure accessible by the processor, such as, for example, preset library 26 of FIG. 2. FIG. 4D shows an example screen shot 130 wherein a single configuration 132 is displayed.

Method 30 proceeds to block 48 when a configuration selection is received from the user. The user may select a configuration and proceed to block 48, for example, by highlighting a configuration and pressing NEXT button 116. The user may also cause method to return to block 42 to select a different probe by pressing BACK button 128.

At block 50 information about the subject of the examination is obtained. Information about the subject may be obtained, for example, by presenting the user with a plurality of data entry fields. FIG. 4E shows an example screen shot 140 wherein a plurality of data entry fields 142 are displayed to the user. In the illustrated example, data entry fields 142 provide spaces for the user to enter the subject's name and, if applicable, identification (ID). Spaces may also be provided for the user's own identification (Operator ID). The user may also enter application-dependent information such as Last Menstruation Period (LMP) and Estimated Delivery Date (EDD). A MORE button 144 may also be provided to allow the user to enter additional information about the subject, the user, and/or the examination to be performed. MORE button 144 may also optionally allow the user to load data about the subject, the user, the examination to be performed and/or information saved from a past examination from a memory of the ultrasound system or from an external data storage medium.

Method 30 proceeds to block 52 when the user presses a START EXAM button 146. In some embodiments, START EXAM button 146 may be unavailable or inoperative until the user has provided at least a minimum threshold of subject information (i.e., the name of the subject or the subject's ID). In other embodiments, start exam button 146 may be operative even if the user has not entered any information about the subject.

In some embodiments, the user may be presented with the option to view training material before starting the examination. The training material may comprise, for example, videos, pictures, or other information related to the selections made by the user. For example, after a user selects the abdominal category and a suitable connected probe for that category, the user may view a video showing the selected probe being used for an Abdominal ultrasound examination. In some embodiments, the user may select a beginner mode wherein training material is automatically displayed after the user has selected the category, the probe, the operational parameters and/or after the user has entered the subject data. In other embodiments, the user may be presented with an option to view training material after making each selection and after entering subject data.

At block 52 the processor activates the selected probe using the set of operational parameters specified by the selected configuration. Method 30 then proceeds to block 54 and ends. The user may then conduct the ultrasound examination to acquire ultrasound imaging data from the subject. The acquired imaging data may be associated with the subject if the user provided subject information at block 50. Method 30 thus allows relatively inexperienced users to conduct an ultrasound examination to acquire imaging data and associate the acquired data with the subject. Method 30 could be used with any types of ultrasound system and probes, and the user need not be familiar with the ultrasound system or the probes in order to conduct the ultrasound examination.

While a number of exemplary aspects and embodiments have been discussed above, those of skill in the art will recognize certain modifications, permutations, additions and sub-combinations thereof.

What is claimed is:

1. A machine implemented method for configuring an ultrasound system for an examination, the method executed by a data processor of the ultrasound system to guide a user through a process for setting up the ultrasound system, the method comprising:
invoking the method in response to the pressing of a dedicated control of a user interface of the ultrasound system;
displaying on a display of the ultrasound system a plurality of examination categories for selection by a user the plurality of examination categories comprising one or more of: Abdominal, Cardiological, General, Gynecological, Musculo-skeletal, Obstetrics, Small Parts and Vascular examination categories;
receiving a user-selection identifying one of the plurality of examination categories as a user-selected examination category by way of the user interface of the ultrasound system;
identifying any probes connected to the ultrasound system by querying the probes themselves or by consulting a data structure stored in memory which is updated to reflect disconnections and reconnections of probes to the ultrasound system;
determining which of the connected probes are suitable for the user-selected examination category and which of the connected probes are not suitable for the user-selected examination category by consulting a data structure stored in memory which indicates probes suitable for each of the plurality of examination categories;
only if at least one suitable probe is connected to the ultrasound system presenting the user with a choice as to which of the at least one suitable connected probes is to be used by displaying on the display of the ultrasound system indicia identifying only the suitable connected probes, thereby preventing the user from selecting for use a probe which is not suitable for the user-selected examination category, the indicia including, for each suitable connected probe a probe icon and a probe name, the probe icon comprising a picture of the suitable connected probe;
receiving user input by way of the user interface of the ultrasound system selecting one of the suitable connected probes as a selected probe;
displaying on the display of the ultrasound system indicia identifying one or more sets of predetermined operational parameters for the selected probe for selection by the user; and,
receiving by way of the user interface user input selecting one of the sets of operational parameters as a selected set of operational parameters;
presenting the user with a plurality of data entry fields for information about a subject of the examination on the display of the ultrasound system;
receiving user input of information about the subject of the examination by way of the user interface;
only if the user has provided at least a threshold amount of information about the subject of the examination making operative a user interface start examination control;
in response to user activation of the start examination control activating selected probe for data acquisition, wherein activating the selected probe comprises applying the user-selected set of operational parameters to the selected probe;
acquiring ultrasound data from the activated probe;
processing the ultrasound image data to yield image data;
displaying the image data on the display in real time; and
associating the ultrasound data with the subject.

2. A method according to claim 1 further comprising presenting to a user the option to view training material before starting an examination, the training material comprising a video demonstrating use of the selected probe for an examination in the user-selected examination category and displaying the video on the display of the ultrasound system.

3. A method according to claim 1 comprising, before displaying the plurality of examination categories:
displaying tutorial material to the user on the display of the ultrasound system, the tutorial material comprising information about how to operate the ultrasound machine.

4. A method according to claim 1 comprising, before activating the selected probe:
displaying training material to the user, the training material comprising information related to at least one of the user-selected examination category and the selected probe.

5. A method according to claim 1, comprising, after the step of determining which of the connected probes are suitable for the user-selected examination category, if none of the connected probes are suitable for the user-selected examination category, prompting the user to connect a suitable probe to the ultrasound system.

6. A method according to claim 1, comprising, after the step of determining which of the connected probes are suitable for the user-selected examination category, if none of the connected probes are suitable for the user-selected examination category, displaying at least one of a help message and an error message.

7. A method according to claim 1 wherein:
the method comprises, prior to receiving the user-selected examination category, providing an opportunity for a user to invoke a tutorial mode; and,
the method comprises, if the tutorial mode is invoked, after reviewing the user-selected probe identification, providing an opportunity for a user to view videos, pictures, or other information selected automatically based on the user-selected examination category and selected probe.

8. A method according to claim 1 comprising receiving input from a user by way of the user interface indicating that the user wishes to select a beginner mode and, in response to the user input operating the ultrasound system in the beginner mode wherein the method comprises automatically displaying training material after one or more of: the user has selected the user-selected category, the user has selected the selected probe, the user has selected the operational parameters and the user has entered the subject data.

9. An ultrasound system configurable to guide a user through a process for setting up the ultrasound system to perform ultrasound examinations, the ultrasound system comprising:
a processor,
a plurality of probe connections, each of the probe connections configured to allow removable connection of an ultrasound probe;
a user interface coupled to the processor, the user interface comprising a dedicated control dedicated for invoking a process for configuring the ultrasound system to perform an ultrasound examination of a subject;
a display coupled to the processor;
a memory accessible by the processor, the memory having computer-readable instructions stored thereon which, when executed by the processor, cause the processor to execute an examination configuration method upon activation of the dedicated control, the examination configuration method comprising:
displaying on a display of the ultrasound system a plurality of examination categories for selection by a user the examination categories comprising one or more of Abdominal, Cardiological, General, Gynecological, Musculo-skeletal, Obstetrics, Small Parts and Vascular examination categories;
receiving a user-selection identifying one of the plurality of examination categories as a user-selected examination category by way of the user interface of the ultrasound system;
identifying any probes connected to the ultrasound system by querying the probes themselves or by consulting a data structure stored in memory which is updated to reflect disconnections and reconnections of probes to the ultrasound system;
determining which of the connected probes are suitable for the user-selected examination category and which of the connected probes are not suitable for the user-selected examination category by consulting a data structure stored in the memory which indicates probes suitable for each of the plurality of examination categories;
only if at least one suitable probe is connected to the ultrasound system presenting the user with a choice as to which of the at least one suitable connected probes is to be used by displaying on the display of the ultrasound system indicia identifying only the suitable connected probes, thereby preventing the user from selecting for use a probe which is not suitable for the user-selected examination category, the indicia including, for each suitable connected probe a probe icon and a probe name, the probe icon comprising a picture of the suitable connected probe;
receiving user input by way of the user interface of the ultrasound system selecting one of the suitable connected probes as a selected probe;
displaying on the display of the ultrasound system indicia identifying one or more sets of predetermined operational parameters for the selected probe for selection by the user; and,
receiving by way of the user interface user input selecting one of the sets of operational parameters as a selected set of operational parameters;
presenting the user with a plurality of data entry fields for information about a subject of the examination on the display of the ultrasound system;
receiving user input of information about the subject of the examination by way of the user interface;
only if the user has provided at least a threshold amount of information about the subject of the examination making operative a user interface start examination control;
in response to user activation of the start examination control activating selected probe for data acquisition, wherein activating the selected probe comprises applying the user-selected set of operational parameters to the selected probe;
acquiring ultrasound data from the activated probe; processing the ultrasound image data to yield image data;
displaying the image data on the display in real time; and
associating the ultrasound data with the subject.

10. An ultrasound system according to claim 9 wherein the memory stores probe information, the probe information comprising a plurality of probe identifications identifying a plurality of probes and, for each of the probes, a plurality of suitability indications, the suitability indications indicating whether or not the probe is suitable for examinations in each of the plurality of examination categories.

11. An ultrasound system according to claim 10 wherein the memory has a preset library stored therein, the preset library comprising the plurality of sets of predetermined operational parameters.

12. An ultrasound system according to claim 11 wherein the memory has a subject database stored therein, the subject database comprising information relating to a plurality of examination subjects.

13. An ultrasound system according to claim 10 wherein the memory has training material stored therein, the training material comprising a plurality of videos, the videos each demonstrating the use of a probe identified by one of the the plurality of probe identifications for an examination in one of the plurality of examination categories.
